(19) United States
(12) Patent Application Publication
(10) Pub. No.: US 2017/0307621 A1
(43) Pub. Date: Oct. 26, 2017

(54) Platform for the identification of tumor-associated cancer/testes antigens

(71) Applicant: Kiromic BioPharma,Inc., Houston, TX (US)

(72) Inventors: Maurizio Chiriva-Internati, Houston, TX (US); Leonardo Mirandola, Houston, TX (US)

(73) Assignee: Kiromic BioPharma, Inc., Houston, TX (US)

(21) Appl. No.: 15/731,143

(22) Filed: Apr. 25, 2017

Related U.S. Application Data
(60) Provisional application No. 62/327,353, filed on Apr. 25, 2016.

(51) Int. Cl. G01N 33/574; C07K 16/30; C12Q 1/68; G06F 19/22

(52) U.S. Cl. CPC G01N 33/57488; G06F 19/22; C07K 16/3053; C12Q 1/6886; C07K 16/3069; C07K 16/3061; C07K 16/30; C07K 16/3046; C07K 16/303; C07K 16/3023; C07K 16/3015; C12Q 2600/158

(57) ABSTRACT

Methods of identifying cancer/testes antigens (CTAs) useful as cancer treatment targets are disclosed and claimed herein. The methods include identifying human sperm proteins to which patients diagnosed with solid or hematological malignancies have established a humoral immune response.

### RELATED APPLICATIONS

The present Utility patent application claims priority to Provisional Application Ser. No. 62/327,353, filed Apr. 25, 2016 and entitled: “Platform For The Identification Of Tumor-Associated Cancer/Testes Antigens”, the contents of which are incorporated herein by reference in their entirety.

### BACKGROUND OF THE INVENTION

Cancerous growth results from the accumulation of mutations that alter the gene expression profiles of normal cells, such as stem cells. Such alteration, in turn makes the cells malignant. This mechanism involves key mutations in tumor-suppressor genes and oncogenes that are expressed and functional in normal cells from which the tumors arise. By contrast, cancer/testes (CT) antigen (CTA) expression is aberrant in the sense that proteins normally restricted to one lineage, are now expressed in a different one. Such aberrant expression is rare in cancer, where lineage fidelity is the general rule. The CTA class provides an exception to this aforementioned rule, in that aberrant expression of CTAs is a common feature in the majority of malignancies.

Aberrant expression of CTAs in cancer cells provides a range of phenotypic traits that, in normal conditions, allow for the survival and function of gametes. These gamete-specific products can be highly advantageous for the cancer cell. When normal stem cells undergoe alteration in the genes that control germ-cell gene expression, a genetic reprogramming occurs; thus leading to tumorigenesis. Specifically, the activation of such a spermatogenesis program can contribute to properties of tumor formation and progression. There are significant corresponding features between cancer cells and germ-cells, which include, but are not limited to: (i) immortalization (which is involved in transformation), (ii) invasion, (iii) induction of meiosis (which can lead to aneuploidy), and (iv) migration (which contributes to metastasis), (v) DNA demethylation, (vi) angiogenesis induction, and (vii) down-regulation of the major histocompatibility complex (immune evasion). See, e.g., Simpson, Caballero, et al. (2005).

Due to the properties of CTAs, there is a marked effort to develop high throughput techniques, such as bioinformatic meta-analyses (see, e.g., Sammut, Feichtinger, et al., (2014)), for the identification of CTAs that may be clinically-relevant as cancer biomarkers and/or immunotherapy targets. Disclosed and claimed in the present patent application is a novel high-throughput technology that may be utilized to identify novel potential CTAs.

### SUMMARY OF THE PRESENT INVENTION

Methods of identifying cancer/testes antigens (CTAs), e.g., immune-reactive peptides and/or sperm-expressed antigens, useful as cancer treatment targets are provided herein. These methods include, but are not limited to, identifying human sperm proteins to which patients diagnosed with solid or hematological malignancies have established a humoral immune response.

By way of non-limiting example, in some embodiments, the disclosed methods include, e.g., a preliminary step of isolating total sperm mRNA from a plurality of sperm cells. In other embodiments, the methods also include sequencing the total sperm mRNA so as to generate total sperm mRNA sequencing data. In some aspects, performing such sequencing includes utilizing whole exome sequencing (WES) on the plurality of sperm cells.

In other embodiments, the methods include comparison of the total sperm mRNA sequencing data to comprehensive human-expressed sequence data in a database so as to identify substantially shared, sperm-expressed coding sequences as a target set of genes. In some embodiments, when the total sperm mRNA sequencing data is not identical to the comprehensive sperm sequence data in an area of a substantially shared, sperm-expressed coding sequence, the total sperm mRNA sequencing data is discarded and the comprehensive sperm sequence data for that area is retained as an identified target set of genes. In other embodiments, the methods include immobilizing peptides corresponding with the target set of genes on a chip, to facilitate subsequent analysis of same.

According to some embodiments of the present invention, the human sperm proteins are identified by methodologies which include, but are not limited to: (a) contacting an addressable array of peptide fragments representing all expressed human sperm protein sequences isolated from the serum of at least one cancer-surviving subject, and/or (b) detecting specific binding of antibodies which are present within the aforementioned at least one serum, to one or more of the immobilized peptides. Additionally, in some aspects of the present invention, the methods include, but are not limited to, the steps of: (c) contacting the addressable array of peptide fragments with serum from at least one normal control subject, and/or (d) detecting specific binding of antibodies, present within the at least one serum, to one or more of the aforementioned immobilized peptides. It should be noted that, in some aspects of the present invention, the identified human sperm proteins are those for which specific binding of antibodies is greater in the cancer-surviving patient serum than in the normal control subject serum. In some embodiments of the present invention, step (c) is conducted concurrently with step (a) and/or step (c) is conducted concurrently with step (b). In various other embodiments of the present invention, the antibodies of the serum isolated from a cancer-surviving subject and antibodies of the serum isolated from the normal control subject are differentially labeled.

According to various aspects of the present invention, the methods include screening the immobilized peptides with sera from a cancer-surviving subject to produce a first signal and screening the immobilized peptides with sera from a healthy subject to produce a second signal. The methods also can include comparing the first signal and the second signal; thereby distinguishing human sperm proteins to which patients diagnosed with solid or hematological malignancies have established a humoral immune response from human sperm proteins to which patients diagnosed with solid or hematological malignancies have not established a humoral immune response.

Also disclosed in the embodiments of the present invention, are methods of treating cancer, such as a solid and/or hematological malignancy. In some embodiments, the methods include: (a) identifying cancer/testes antigens (CTAs) comprising human sperm proteins to which patients diagnosed with solid or hematological malignancies have established a humoral immune response; (b) generating anti-CTA polyclonal antibodies against the identified CTAs; and/or (c) administering a therapeutically effective amount of the antibodies to the subject and thereby eliciting an autologous immune response in the subject in a manner sufficient to treat the solid or hematological malignancies.

In such aspects wherein the cancer is a hematological malignancy, the malignancy can be any one or a combination of: (i) T-cell acute lymphocytic leukemia, (ii) T-cell acute lymphoblastic leukemia, (iii) T-cell chronic lymphocytic leukemia, (iv) non-Hodgkin lymphomas, (v) Hodgkin lymphoma, (vi) multiple myeloma, (vii) plasma cell leukemia, (viii) B-cell acute lymphocytic leukemia, (ix) B-cell acute lymphoblastic leukemia, (x) chronic myelogenous leukemia (CML), and/or (xi) acute myeloid leukemia (AML).

### BRIEF DESCRIPTION OF THE DRAWINGS

This patent application or patent application file consists of at least one drawing executed in color. Copies of this patent or patent application with color drawing(s) will be provided by the Office upon request and payment of the required fee.

FIG. 1—discloses that cancer/testis antigen (CTAs) are immunogenic proteins that have been reported to induce both humoral and cell-mediated immune responses in patients without the induction of toxicities. The Applicant's have developed a novel selection Biomarker Lead Antigen Discovery Engine (i.e., BLADE™) platform utilizing a high-throughput system intended to detect immunogenic CTAs using the patient's serum. As a source of new potential CTA sequences, BLADE™ uses the testis gene expression signature.

The aberrant expression of CTAs in cancer cells provides a range of phenotypic traits that in normal conditions allow for the survival and function of gametes. A systematic evaluation of spermatogenesis-related proteins as possible CTA family members has, heretofore, never been done.

For the first selection methodology (BLADE™ prototype), CTAs were prioritized basing upon two key criteria: (i) Specificity—only testis genes from the tissue enriched panel, i.e., with high specificity scores (FKPM data by the Human Tissue Atlas) were included; and (ii) Prevalence—high-specificity score testis genes were prioritized according to their expected prevalence across multiple tumor types.

FIG. 2—sets forth the development of the Applicant's proprietary Biomarker Lead Antigen Discovery Engine (BLADE™) platform is based upon a high-density peptide array, consisting of overlapping peptide libraries obtained from the sequence of each CTA included in the panel. Expression and immunogenicity is evaluated at the same time, by incubating the array with a few microliters of serum, to measure the presence of peptide-specific antibodies. Comparison of signal intensities with that of healthy control serum allows for determination of specificity.

Of important note, the measurement of circulating (i.e., humoral) antibodies against tumor antigens is a powerful tool in determining their immunogenicity, as has previously been shown by the Inventors.

Candidate CTAs are then evaluated against a panel of healthy tissues and in a sample of the patient's tumor. When selective tumor expression is confirmed, the identified B-cell epitope can be directly used to design antibody therapies, while the CTA peptide library is used to identify T-cell epitopes.

FIG. 3—graphically-illustrates: (i) the percent seropositivity of various samples by the type of tumor; and (ii) antigen-ranking and prioritization based upon overall percent of positive samples across different histological types.

BLADE™ data were analyzed by Log₁₀ conversion of the resulting measured fluorescence units. Positivity cutoff was set for each peptide, as the average of the healthy controls plus three-times the standard deviation of the healthy control group. Each peptide showing immunoreactivity in one or more healthy control sera, based on said cutoff, was excluded from the analysis. Samples showing high background were also excluded from the analysis.

Types of cancer tissues analyzed in these series of experiments include: (i) non-small cell lung carcinoma (NSCLC); (ii) lymphoma (iii) prostate; (iv) pancreatic; (v) colorectal; (vi) thyroid; (vii) cervical; (viii) ovarian; (ix) melanoma; and (x) head and neck squamous cell carcinoma (HNSCC).

FIG. 4—represents in tabular form the percent of sero-positive cancer tissue samples. In the Table, columns represent the cancer types; whereas the rows identify the specific antigen type utilized.

FIGS. 5(a) and 5(b)—graphically-illustrates the percent seropositivity of various samples by the type of tumor as screened by different antigens. BLADE™ data were analyzed by Log₁₀ conversion of the resulting measured fluorescence units. Positivity cutoff was set for each peptide, as the average of the healthy controls plus three-times the standard deviation of the healthy control group. Each peptide showing immunoreactivity in one or more healthy control sera, based on said cutoff, was excluded from the analysis. Samples showing high background were also excluded from the analysis.

Types of cancer tissues analyzed in these series of experiments include: (i) non-small cell lung carcinoma (NSCLC); (ii) lymphoma (iii) prostate; (iv) pancreatic; (v) colorectal; (vi) thyroid; (vii) cervical; (viii) ovarian; (ix) melanoma; (x) head and neck squamous cell carcinoma (HNSCC); and (xi) central nervous system.

### DETAILED DESCRIPTION OF THE PRESENT INVENTION

Methods of identifying cancer/testes antigens (CTAs) useful as cancer treatment targets are provided herein. The methods include, but are not limited to, identifying human sperm proteins to which patients diagnosed with solid or hematological malignancies have established a humoral immune response.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which the present invention belongs. Although any methods and materials similar or equivalent to those described herein can be used in the practice or testing of the present invention, some potential and exemplary methods and materials may now be described.

In addition, any and all publications mentioned herein are incorporated herein by reference to disclose and describe the methods and/or materials in connection with which the publications are cited. It is understood that the present disclosure supersedes any disclosure of an incorporated publication to the extent there is a contradiction. The publications discussed herein are provided solely for their disclosure prior to the filing date of the present patent application. Further, the dates of publication provided may be different from the actual publication dates which may need to be independently confirmed. To the extent such publications may set forth definitions of a term that conflict with the explicit or implicit definition of the present disclosure, the definition of the present disclosure controls.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### 1.1 Definitions

The terms utilized in the present Claims and Specification are defined as set forth below, unless otherwise specified.

As utilized herein, the term “amino acid” refers to natural amino acids, unnatural amino acids, and amino acid analogs, all in their D- and L-stereoisomers, unless otherwise indicated, if their structures allow such stereoisomeric forms.

As utilized herein, the term “natural amino acids” are: alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V).

As utilized herein, the term “unnatural amino acids” include, but are not limited to, azetidinecarboxylic acid, 2-aminoadipic acid, 3-aminoadipic acid, beta-alanine, naphthylalanine (“naph”), aminopropionic acid, 2-aminobutyric acid, 4-a 5 minobutyric acid, 6-aminocaproic acid, 2-aminoheptanoic acid, 2-aminoisobutyric acid, 3-aminoisobutyric acid, 2-aminopimelic acid, tertiary-butylglycine (“tBuG”), 2,4-diaminoisobutyric acid, desmosine, 2,2′-diaminopimelic acid, 2,3-diaminopropionic acid, N-ethylglycine, N-ethylasparagine, homoproline (“hPro” or “homoP”), hydroxylysine, allo-hydroxylysine, 3-hydroxyproline (“3Hyp”), 4-hydroxyproline (“4Hyp”), isodesmosine, allo-isoleucine, N-methylalanine (“MeAla” or “Nime”), Nalkylglycine (“NAG”) including N-methylglycine, N-methylisoleucine, N-alkylpentylglycine (“NAPG”) including N-methylpentylglycine. N-methylvaline, naphthylalanine, norvaline (“Norval”), norleucine (“Norleu”), octylglycine (“OctG”), ornithine (“Orn”), pentylglycine (“pG” or “PGly”), pipecolic acid, thioproline (“ThioP” or “tPro”), homoLysine (“hLys”), and homoArginine (“hArg”).

As utilized herein, the term “amino acid analog” refers to a natural or unnatural amino acid where one or more of the C-terminal carboxy group, the N-terminal amino group, and the side-chain functional group has been chemically blocked (either reversibly or irreversibly) or otherwise modified to another functional group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide and S-(carboxymethyl)-cysteine sulfone.

As utilized herein, the term “peptide” refers a short polymer of amino acids linked together by peptide bonds. In contrast to other amino acid polymers (e.g., proteins, polypeptides, etc.), peptides are of about 50 amino acids or less in length. A peptide may comprise natural amino acids, non-natural amino acids, amino acid analogs, and/or modified amino acids. A peptide may be a subsequence of naturally occurring protein or a non-natural (synthetic) sequence.

As used herein, a “conservative amino acid substitution” refers to the substitution of an amino acid in a peptide or polypeptide with another amino acid having similar chemical properties, such as size or charge. For purposes of the present disclosure, each of the following eight groups contains amino acids that are conservative substitutions for one another: (i) Alanine (A) and Glycine (G); (ii) Aspartic acid (D) and Glutamic acid (E); (iii) Asparagine (N) and Glutamine (Q); (iv) Arginine (R) and Lysine (K); (v) Isoleucine (I), Leucine (L), Methionine (M), and Valine (V); (vi) Phenylalanine (F), Tyrosine (Y), and Tryptophan (W); (vii) Serine (S) and Threonine (T); and (viii) Cysteine (C) and Methionine (M).

As utilized herein, a “semi-conservative amino acid substitution” refers to the substitution of an amino acid in a peptide or polypeptide with another amino acid within the same class. Naturally occurring residues may be divided into classes based upon their common side chain properties, for example: (i) polar positive (histidine (H), lysine (K), and arginine (R)); (ii) polar negative (aspartic acid (D), glutamic acid (E)); (iii) polar neutral (serine (S), threonine (T), asparagine (N), glutamine (Q)); (iv) non-polar aliphatic (alanine (A), valine (V), leucine (L), isoleucine (I), methionine (M)); and (v) non-polar aromatic (phenylalanine (F), tyrosine (Y), tryptophan (W)); proline and glycine; and cysteine.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis, rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs. Non-conservative substitutions may involve the exchange of a member of one class for a member from another class.

As utilized herein, the term “non-conservative amino acid substitutions” may involve the exchange of a member of one class for a member from another class.

The term “protein” is utilized herein regardless of length, and is used synonymously with the term “polypeptide.”

As utilized herein, the terms “peptide mimetic” or “peptidomimetic” refer to a peptide-like molecule that emulates a sequence derived from a protein or peptide.

A peptide mimetic or peptidomimetic may contain amino acids and/or non-amino acid components. Examples of peptidomimetics, include chemically modified peptides, including: peptoids (i.e., the side chains are appended to the nitrogen atom of the peptide backbone, rather than to the α-carbons), β-peptides (i.e., the amino group bonded to the β carbon rather than the α carbon), and the like.

As utilized herein, the terms “administration of” or “administering” a therapeutic compound or a composition refer to introducing the compound or composition into the body of a subject in need of treatment. The term includes directly introducing the compound or composition into the subject's body, such as by parenteral administration, or indirectly introducing the compound or composition, for example by prescribing that the compound or composition be introduced into the subject's body, by ordering that the compound or composition be introduced into the subject's body, or by providing the compound or composition for use by the subject in accordance with instructions or advice. Administration of or administering includes administration by any route of administration, including: oral administration; buccal administration; sublingual administration; parenteral administration (e.g., including intravenous, intramuscular, subcutaneous, intraperitoneal, intrathecal, and intracerebroventricular administration); intratumoral administration; nasal administration; pulmonary administration; and rectal administration.

As utilized herein, the term “subject” broadly refers to any animal, including but not limited to, human and non-human animals. In typical embodiments, the subject is a mammal, including a human. The term “human” includes human subjects of either sex and at any stage of development (e.g., fetuses, neonates, infants, juveniles, adolescents, adults). As utilized herein, the term “patient” refers to a human subject who is being treated for a disease or condition.

As utilized herein, the term “effective amount” refers to the amount of the subject compound or composition sufficient to elicit a desired biological response in a cell, tissue, or organism.

As utilized herein, the term “therapeutically effective amount” refers to the amount of the subject compound or composition sufficient to provide a therapeutic or clinical benefit to the subject. The therapeutic or clinical benefit may include alleviation of symptoms, reduction in the severity of the disease, slowing disease progression, stopping disease progression, increasing overall survival, increasing progression-free survival, increasing efficacy of other therapies. An effective amount or therapeutically effective amount can be administered in one or more administrations, applications or dosages and is not intended to be limited to a particular formulation or administration route.

As utilized herein, the terms “treatment” or “treating” means obtaining a beneficial or intended clinical result. The beneficial or intended clinical result may include alleviation of symptoms, reduction in the severity of the disease, slowing disease progression, stopping disease progression, increasing overall survival, increasing progression-free survival, increasing efficacy of other therapies.

As utilized herein, the term “pharmaceutically acceptable” component is one that is suitable for use with subjects, e.g., humans and/or animals, without undue adverse side effects (such as toxicity, irritation, and allergic response) commensurate with a clinical benefit/risk ratio.

As utilized herein, the terms “resistant” or “refractory” when referring to a cancer mean that the cancer cells are no longer responsive to a prior chemotherapeutic or other treatment regimen, such as radiation therapy. The terms “sensitize” or “resensitize” when referring to a cancer means that the cancer cells again become responsive to a prior chemotherapeutic or other treatment regimen, such as radiation therapy, to which they had become resistant or refractory.

As utilized herein, the term “sequence identity” with respect to peptides and polypeptides refers to the degree to which two peptide or polypeptide sequences have the same sequential composition of monomer subunits. The “percent sequence identity” is calculated by: (1) comparing two optimally aligned sequences over the length of the shorter sequence; (2) determining the number of positions containing identical amino acids to yield the number of matched positions, (3) dividing the number of matched positions by the total number of positions in the shorter sequence, and (4) multiplying the result by 100 to yield the percent sequence identity.

As utilized herein, the term “sequence similarity” refers to the degree with which two peptide or polypeptide sequences differ only by conservative and/or semi-conservative amino acid substitutions, as context dictates. The “percent sequence similarity” is calculated by: (1) comparing two optimally aligned sequences over the length of the shorter sequence; (2) determining the number of positions containing (a) identical monomers and (b) conservative and/or semi-conservative substitutions, as context dictates, to yield the number of “similar” positions; (3) dividing the number of similar positions by the total number of positions in the shorter sequence; and (4) multiplying the result by 100 to yield the percent sequence identity or percent sequence similarity.

### 1.2 CTAs as a Source of Antigens

Provided herein are methods of identifying useful cancer treatment targets. Specifically, the targets are CTAs, such as sperm proteins (e.g., human sperm proteins) to which patients diagnosed with solid or hematological malignancies have established a humoral immune response. In particular, high-throughput methodologies for identifying sperm-expressed antigens which are suitable for utilization in cancer immunotherapies and/or as cancer biomarkers are disclosed. As disclosed herein, the methods include increasing the number of available target antigens in cancer vaccines. In some aspects of the methods, CTAs are immune-reactive peptides. Also, according to various embodiments, CTAs are sperm-expressed antigens.

According to various embodiments, effective targets, such as CTAs, elicit strong immunogenicity and/or demonstrate selective expression in cancer cells, but not in normal tissues (see, Mellman, Coukos, et al. (2011)). CTAs are particularly effective in this regard due to their tumor-restricted expression pattern and immunogenicity (see, Caballero and Chen (2009); Mirandola, et al. (2011); Gjerstorff, Andersen, et al. (2015)).

### 1.3 Identifying CTAs of Interest

In some aspects of the embodiments, the methods include a step, e.g., a preliminary step, of isolating total sperm nucleic acid (e.g., RNA, DNA, human total sperm mRNA) from a plurality of sperm cells, such as cells in a sperm sample.

1.3.1 Isolation of Sperm Total Nucleic Acid

In various embodiments, the methods include isolating total sperm nucleic acid (e.g., RNA). Isolating total sperm RNA may be achieved by performing an isolation protocol which includes any one or a combination of the following listed steps:

Diluting PureSperm to 50% with a buffer, such as PureSperm buffer (Spectrum Technologies); equilibrating to room temperature (at least 10-15 minutes); and/or aliquoting 3 mL of 50% PureSperm per sample (1.5 mL PureSperm and 1.5 mL PureSperm buffer) into individual 15 mL conical tubes. Allowing fresh ejaculate samples, including a plurality of sperm cells, to liquefy at room temperature for approximately 30 minutes. If the ejaculate sample is frozen, the methods include thawing said sample rapidly by holding in the hand until liquefied.

Transferring the sample to a 15 mL conical tube and adding 6-10 mL of PureSperm buffer to wash sample by, for example, gently inverting the tube a plurality of times. Using a hemocytometer to estimate the total number of cells. Centrifuging for 15 minutes at 4° C. at 300×g, and then discarding the supernatant into a 10% bleach solution. Re-suspending the cell pellet in 1 mL of PureSperm buffer. Overlaying the sample onto a 50% PureSperm step-gradient using a transfer pipette while being careful not to disturb the interface.

Disengaging the rapid acceleration and brake options from the centrifuge. Centrifuging the gradient for 20 minutes at room temperature at 200×g. Removing the pellet from the conical tube by directing the tip of transfer pipette to the bottom of the tube. Extracting the sperm pellet with as little liquid as possible.

Transferring the sperm pellet to a new conical tube and resuspend in 10 mL PureSperm buffer. Washing the cells by inverting the tube several times. Centrifuging for 15 minutes at 4° C. at 300×g. Discarding the supernatant into a 10% bleach solution.

While the purified sperm are being collected by centrifugation, preparing the materials required for cell lysis. Preparing one homogenization tube for each sample. To a 2 mL microcentrifuge tube, adding 500 μL RLT buffer (QIAGEN RNeasy Kit), 7.5 μL β-mercaptoethanol, and 100 mg of 0.2 mm nuclease-free stainless steel beads.

After centrifugation, decanting the supernatant and resuspending the collected sperm pellet in any residual liquid. Adding up to 100 million cells to the previously prepared homogenization tube. If the sample is greater than 100 million cells, dividing it into equal aliquots that are less than 100 million cells each and treating said aliquots as separate samples until the extraction process is complete.

Placing the homogenization tube in a Disruptor Genie (Scientific Industries) and shaking for 5 minutes. Adding 500 μL of Qiazol (QIAGEN) and shaking for 2 minutes with the Disruptor Genie, as set forth above. Adding 200 μL of chloroform and rapidly shaking by hand for approximately 15 seconds. Letting the mixture sit at room temperature for 3-5 minutes.

Centrifuging for 20 minutes at 4° C. at 12,000×g. Removing the upper aqueous layer using a wide bore pipette tip and placing the recovered aqueous layer into a 2 mL tube. Extracting RNA using RNeasy (QIAGEN) protocol as follows below.

Adding 360 μL of 100% ethanol for every 500 μL of upper aqueous phase and mix the solution by pipetting up-and-down. Applying 700 μL of the sample (including any precipitate that forms) to an RNeasy mini column that is placed in a 2 mL collection tube. Closing tube and allowing the fluid to flow through the RNeasy mini column by centrifugation at ≧8,000×g for 15 seconds. Removing the flow-through (FT) containing the small RNA fraction and place the small RNA fraction in a 15 mL conical tube. Repeating these aforementioned steps with any remaining sample.

After the entire sample has been centrifuged through the RNEasy mini column and the FT collected, placing the columns containing the large RNAs at 4° C. Extracting the small RNA first and then complete the extraction of the large RNA (see, step of “removing the columns” below). Adding 0.65 volumes of 100% ethanol to the 15 mL conical tube containing the small RNA FT, and mixing thoroughly by vortexing, but not centrifuging.

Transferring 700 μL to an RNeasy MinElute spin column placed in a 2 mL collection tube. Centrifuging for 15 seconds at ≧8,000×g. Discarding the FT. Repeating the previous step until the entire sample has passed through the RNeasy MinElute spin column. Discarding the FT each time in a guanidinium waste container.

Adding 500 μL RPE wash buffer to the RNeasy MinElute spin column. Centrifuging for 15 seconds at ≧8,000×g and discarding the FT. Adding 500 μL of 80% ethanol to the RNeasy MinElute spin column. Centrifuging for 15 seconds at ≧8,000×g. Discarding the FT and the collection tube.

Placing the RNeasy MinElute spin column in a new 2 mL collection tube. Centrifuging for 1 minute at ≧8,000×g. Placing the RNeasy MinElute spin column in a 1.5 mL collection tube. Adding 14 μL of RNase-free water directly to the spin column membrane. Centrifuging for 1 minute at ≧8,000×g to elute the small RNA. Repeating with the previous step with another 14 μL of of RNase-free water. Adding 1 μL of RNase Block (Stratagene) and DTT to a final concentration of 10 mM. Storing at −80° C.

Removing the columns containing the large RNAs from the temporary 4° C. storage. Adding 700 μL of RW1 buffer to the RNeasy column. Centrifuging at ≧8,000×g. Discarding the FT and collection tube. Transferring the RNeasy column to a new collection tube.

Pipetting 500 μL RPE buffer onto the RNeasy column. Centrifuging at ≧8,000×g, for 15 seconds and discarding the FT.

Loading another 500 μL of RPE buffer onto the RNeasy column. Centrifuging for 2 minutes at ≧8,000×g so as to dry the RNeasy column silica-gel membrane. Transferring the RNeasy column to another tube and centrifuging again, as above, for 1 minute to remove any traces of ethanol.

Transferring the RNeasy column to the final 1.5 mL collection tube. Eluting the large RNA fraction with 50 μL of RNase-free water. Centrifuging at 8,000×g for 1 minute and then repeating the elution with an additional 50 μL of RNase-free water. Adding 1 μL of RNase Block (Stratagene) and adding DTT (to a final concentration of 10 mM) to the 100 μL volume final RNA sample. Storing a solution, including isolated total sperm RNA, at a temperature of −80° C.

1.3.2 Preparation of Total Sperm Nucleic Acid Library and Library Sequencing

According to some aspects, the methods include preparing a total sperm nucleic acid library. For example, a total sperm DNA or RNA (e.g., mRNA) library. A total sperm mRNA library (SpeRNA library) may be prepared according to the embodiments disclosed herein. SpeRNA sequencing may also be performed. In various embodiments, total RNA (25 ng) is prepared as detailed above. The total RNA is then used to prepare an mRNA library, for example, by using a kit such as the Illumina® TruSeq Stranded mRNA Library Prep Kit® for NeoPrep™.

The methods also include preparing and performing nucleic acid library sequencing, e.g., SpeRNA high-throughput sequencing. Such methods can include sequencing the total sperm mRNA to generate total sperm nucleic acid (e.g., mRNA) sequencing data. Such data may include: (i) the sequence of nucleic acids; and/or (ii) the amount; and/or (iii) the location of variation as compared to other sequences. Sequencing according to the subject methods may include performing whole exome sequencing (WES) on a plurality of sperm cells in a sample.

The methods, in various embodiments, also include providing instructions to prepare a total sperm nucleic acid library. The subject methods also may include providing instructions to prepare and/or perform nucleic acid library sequencing (e.g., SpeRNA high-throughput sequencing).

1.3.3 Identifying Target Sets of Genes

The methods, in some aspects, include comparing the total sperm mRNA sequence to a comprehensive human expressed sequence to identify substantially shared, sperm-expressed coding sequences as a target set of genes. As used herein, the term “substantially” means to a great or significant extent, such as almost fully or almost entirely.

In some embodiments the methods include comparing the total sperm mRNA sequencing data to comprehensive human expressed sequence data in a database to identify substantially shared sperm-expressed coding sequences as a target set of genes. The comparison can be performed automatically by a central processing unit, such as a central processing unit including the database. In some aspects of the present invention, when the total sperm mRNA sequencing data is not identical to the comprehensive sperm sequence data in an area of a substantially shared sperm-expressed coding sequence, the total sperm mRNA sequencing data is discarded and the comprehensive sperm sequence data for that area is retained as an identified target set of genes.

According to other embodiments of the present invention, sequencing results from the previous step are run through the Basic Local Alignment Search Tool (BLAST). Specifically, the search can be run against the Homo sapiens ESTs database using the Megablast algorithm for highly similar sequences, to identify sperm-expressed coding sequences.

1.3.4 Synthesis of SpeRNA-Coded Peptides

In some embodiments of the present invention, the methods include immobilizing peptides, that correspond with the target set of genes, on a chip. According to the disclosed embodiment, the corresponding protein sequence can be retrieved for each identified target gene or set of genes, and then a library of overlapping peptides can be created. Such a library may be created using the following algorithm; wherein amino acid positions are numbered from the N-terminus to the C-terminus of the protein.

(A) Peptide #1 in the library corresponds to the first 15 amino acids; (B) peptide #2 starts at amino acid number 11 (which is included) and extends to amino acid number 25 (which is also included); and therefore peptides #1 and #2 overlap in 5 amino acids (C). In general, each peptide #n starts at the start position of peptide #(n−1)+10 and ends at the end position of peptide #(n−1)+10.

In some aspects, a 15 amino acid length distribution is applied. Such a distribution is applied, for example, because the typical length distribution of antibody epitopes ranges from 5 to 15 amino acids (see, Singh, Ansari, et al. (2013)). Such a procedure will generate n=protein length/10 peptides for each identified proteins. Because a human protein is, on average, 480-amino acid in length, each peptide library consists of approximately 50 peptides. The human sperm proteome is estimated to include about 5,000 proteins (see, Wang, Guo, et al. (2013)). Therefore, a total of 250,000 peptides is expected. Customizable, high-density peptide chips that are commercially-available can contain up to 100,000 individual peptides per chip (see, Buus, Rockberg, et al. (2012)); therefore with only three of such high-density chips, the entire library can be screened.

In various embodiments of the present invention, each array is generated within a small (ca. 1×2 cm) rectangular area on a functionalized microscope slide. The individual peptide fields are square-shaped with a freely definable side length—varying from 10×10 and up to 1000×1000 μm. In various aspects, larger fields are used (e.g., 20×20 μm) fields to facilitate recording of the arrays after binding of ligands.

1.3.5 Hybridization with Sera and Identification of Immune-Reactive Peptides

In some embodiments of the present invention, the methods include hybridizing with sera and/or identifying immune-reactive peptides. According to various embodiments, the entire processes of hybridization and/or detection can be outsourced (e.g., Schafer-N ApS). As such, in some embodiments, the methods include providing instructions for hybridization with sera and/or detection of immune-reactive peptides.

In some aspects, human sperm proteins are identified by contacting an addressable array of peptide fragments representing all expressed human sperm protein sequences with serum isolated from at least one cancer-surviving subject. According to other embodiments, the methods include screening the immobilized peptides with sera from a cancer-surviving subject to produce a first signal. The methods also, in some embodiments, include detecting specific binding of antibodies present within the at least one serum to one or more of the aforementioned immobilized peptides.

The methods also may include contacting the addressable array of peptide fragments with serum from at least one normal control subject, e.g., a subject which is not a cancer-surviving subject. Also, according to some embodiments, the methods include screening the immobilized peptides with sera from a healthy subject to produce a second signal. In some aspects, the methods include detecting specific binding of antibodies present within the at least one serum to one or more of the immobilized peptides. In some embodiments, the identified human sperm proteins are those for which specific binding of antibodies is greater in the cancer-surviving patient serum than in the normal control subject serum.

In some aspects, a step of contacting an addressable array of peptide fragments representing all expressed human sperm protein sequences with serum from at least one cancer-surviving subject is conducted concurrently with a step of contacting the addressable array of peptide fragments with serum from at least one normal control subject. Also in some aspects, a step of contacting an addressable array of peptide fragments representing all expressed human sperm protein sequences with serum from at least one cancer-surviving subject, is conducted concurrently with a step of detecting specific binding of antibodies present within the at least one serum to one or more of the immobilized peptides. Additionally, according to embodiments of the disclosed methods, the antibodies of the serum from cancer-surviving subject and antibodies of the serum from the normal control subject are differentially labeled.

1.3.6 Identification of Positive Peptides and Genes of Origin

In some aspects of the methods of the present invention, such as methods which include screening the immobilized peptides with sera isolated from a cancer-surviving subject to produce a first signal and/or screening the immobilized peptides with sera isolated from a healthy subject to produce a second signal; and wherein these aforementioned methods also may include comparing the first signal and second signal and thereby allow distinguishing human sperm proteins to which patients diagnosed with solid or hematological malignancies have established a humoral immune response from those human sperm proteins to which patients diagnosed with solid or hematological malignancies have not established a humoral immune response.

Also, as noted above, according to some aspects of the methods disclosed herein, the antibodies of the serum isolated from cancer-surviving subject and the antibodies of the serum isolated from a normal control subject are differentially labeled. As such, in some aspects, the methods include distinguishing these differentially labeled antibodies.

In other aspects, the methods disclosed in the present invention include diagnosing a patient with cancer, such as a specific type of cancer. Analyzed sera, in some aspects of the methods, are from subjects newly diagnosed with solid or hematological malignancies. Each cancer diagnosis can be analyzed separately. In some aspects, normal control groups are different for each tumor group. Sera isolated from cases, e.g., tumor-diagnosed subjects and controls, e.g., individuals with no known malignant condition, are collected by consecutive sampling according to the disclosed methods. In some aspects, 100 or more cases and/or 100 or more controls are analyzed for each cancer type.

According to the subject methods, signal intensities for each peptide found in each serum are recorded. Values from cases and controls groups may then be plot separately and statistically significant differences may be evaluated by use of, e.g., the Mann-Whitney test (α=0.05). Before performing the analysis, outliers may be identified by the extreme studentized deviate (ESD) method or the Dixon's ratio test, and removed.

Peptides with calculated p<0.05 value are included in the new CTA panel and, according to various aspects, may be further tested using: (i) tetramer staining to identify naturally occurring, circulating, and/or tumor-infiltrating peptide-specific T-cells; (ii) cytotoxicity of in vitro generated CTL using autologous dendritic cell-PBL co-cultures; and/or (iii) in vivo murine models of the tumors of interest using murine homologues of the peptides sequence.

For T-cell based assays, each peptide can be used “as is” or corresponding T-cell specific immune-dominant peptides can be derived from the protein containing the peptide originally identified using the disclosed methods.

Furthermore, according to the subject embodiments, a functional validation of identified antigen may be performed using the whole antigen instead of the identified peptide(s). If more than one peptide is identified for each protein, the functional analysis will prioritize peptides with highest affinity score for the binding to class I MHC molecules, using the NetMHC 4.0 Server for the prediction of peptide-MHC class I binding using artificial neural networks.

1.3.7 Methods of Treating Cancer Using Identified CTAs

The disclosed methods also include treating cancer in a subject. In various aspects, such methods include identifying cancer/testes antigens (CTAs) according to any of the methods disclosed herein.

As noted above, in various embodiments, the cancer/testes antigens CTAs include human sperm proteins to which patients diagnosed with solid or hematological malignancies have established a humoral immune response. In various embodiments, the hematological malignancy is selected from: T-cell acute lymphocytic leukemia, T-cell acute lymphoblastic leukemia, T-cell chronic lymphocytic leukemia, non-Hodgkin lymphomas, Hodgkin lymphoma, multiple myeloma, plasma cell leukemia, B-cell acute lymphocytic leukemia, B-cell acute lymphoblastic leukemia, chronic myelogenous leukemia (CML), and acute myeloid leukemia (AML).

In some aspects, the methods include generating anti-CTA polyclonal antibodies against the identified CTAs. Such antibodies may, in turn, be administered in a therapeutically-effective amount to the subject. Such administration may elicit an autologous immune response in the subject in a manner sufficient to treat the cancer.

In various aspects of the disclosed methods, the epitope(s) targeted by an antibody are identified and characterized; thereby establishing antibody reactivity, highlighting possible cross-reactivities, and/or warning against unwanted (e.g., autoimmune) reactivities. In certain embodiments, antibodies target proteins as either conformational or linear epitopes. The latter can be probed with peptides.

To perform high-throughput, high-resolution mapping of linear antibody epitopes, ultra high-density peptide microarrays, generating several hundred thousand different peptides per array, may be applied. Using exhaustive length and substitution analysis, the specificity of a panel of polyclonal antibodies raised against linear epitopes of the human proteome may be achieved according to the methods disclosed herein and detailed descriptions of the involved specificities may thus be examined.

The epitopes identified may range, for example, from 4 to 12 amino acids in length. The antibodies can have considerable specificity, frequently disallowing even single conservative substitutions. In various embodiments, multiple distinct epitopes are identified for the same target protein, thus suggesting an efficient approach to the generation of paired antibodies. Epitope mapping approaches can identify similar, although not necessarily identical, epitopes. As such, ultra high-density peptide microarrays can be used according to the methods for linear epitope mapping. With an upper theoretical limit of 2,000,000 individual peptides per array, such peptide microarrays may even be used for a systematic validation of antibodies at the proteomic level, according to the subject methods.

### 2. EXAMPLES

The following examples are set forth so as to provide those individuals of ordinary skill in the art with a complete disclosure and description of how to make and use the present invention, and are not intended to limit the scope of what the inventors regard as their invention, nor are they intended to represent that the experiments below are all or the only experiments actually performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric (i.e., 14.7 psi).

### Example 1: Isolation of Human Sperm Total RNA

In a first step, the methods can include isolating total sperm nucleic acid, e.g., RNA. In these methods, isolating total sperm RNA was achieved by performing an isolation protocol including any one or combination of the following steps:

(1) Dilute PureSperm to 50% with PureSperm buffer (Spectrum Technologies). Equilibrate to room temperature (at least 10-15 minutes). Aliquot 3 mL of 50% PureSperm per sample (1.5 mL PureSperm and 1.5 mL PureSperm buffer) into individual 15 mL conical tubes.
(2) Allow fresh ejaculate samples to liquefy at room temperature for 30 minutes. If liquefied sample was frozen thaw sample rapidly by hand. (3) Transfer the sample to a 15 mL conical tube and add 6-10 mL of PureSperm buffer to wash sample by inverting the tube several times. (4) Using a hemocytometer estimate the total number of cells. (5) Centrifuge for 15 minutes at 4° C. at 300×g, and then discard the supernatant into a 10% bleach solution.
(6) Resuspend the cell pellet in 1 mL of PureSperm buffer. (7) Overlay the sample onto a 50% PureSperm step-gradient using a transfer pipette, using care so as to not to disturb the interface. (8) Disengage the rapid acceleration and brake options from the centrifuge. Centrifuge the gradient for 20 minute at room temperature at 200×g. (9) Remove the pellet from the conical tube by directing the tip of transfer pipette to the bottom of the tube. Extract the sperm pellet with as little liquid as possible. (10) Transfer the sperm pellet to a new conical tube and resuspend in 10 mL PureSperm buffer.
(11) Wash the cells by inverting the tube several times. (12) Centrifuge for 15 minutes at 4° C. at 300×g. (13) Discard the supernatant into a 10% bleach solution. (14) As the purified sperm are being collected by centrifugation, prepare the materials required for cell lysis. Prepare one homogenization tube for each sample. To a 2 mL microcentrifuge tube, add 500 μL RLT buffer (QIAGEN RNeasy Kit), 7.5 μL β-mercaptoethanol, and 100 mg of 0.2 mm nuclease-free stainless steel beads. (15) After centrifugation, decant the supernatant and resuspend the sperm pellet in any residual liquid. (16) Add up to 100 million cells to the homogenization tube prepared in Step 14. If the sample is greater than 100 million cells, it should be divided into equal portions that are less than 100 million cells and treated as separate samples until the extraction is complete. (17) Place the homogenization tube in a Disruptor Genie (Scientific Industries) and shake for 5 minutes. (18) Add 500 μL of Qiazol (QIAGEN) and shake for 2 minutes with the Disruptor Genie as disclosed above. (19) Add 200 μL of chloroform and rapidly shake by hand for 15 seconds. (20) Let the mixture sit at room temperature for 3-5 minutes. (21) Centrifuge for 20 minutes at 4° C. at 12,000×g. (22) Remove the upper aqueous layer using a wide bore pipette tip and place the recovered aqueous layer into a 2 mL tube.

(23) Extract RNA using RNeasy (QIAGEN) protocol as follows. (24) Add 360 μL of 100% ethanol for every 500 μL of upper aqueous phase and mix by pipetting up-and-down. (25) Apply 700 μL of the sample, including any precipitate, to an RNeasy mini column that is placed in a 2 mL collection tube. Close tube and allow the fluid to flow through the column by centrifugation at ≧8,000×g for 15 seconds. (26) Remove the flow-through (FT) containing the small RNA fraction and place said small RNA fraction in a 15 mL conical tube. Repeat with any remaining sample. (27) After the entire sample has been centrifuged through the RNEasy column and the FT collected, place the columns containing the large RNAs at 4° C. Continue to Step 28 to initially extract the small RNA and then complete the extraction of the large RNA (see, Step 37). (28) Add 0.65 volumes of 100% ethanol to the 15 mL conical tube containing the small RNA FT, and mix thoroughly by vortexing. Do not centrifuge. (29) Transfer 700 μL to an RNeasy MinElute spin column placed in a 2 mL collection tube. (30) Centrifuge for 15 seconds at ≧8,000×g. Discard the FT.

(31) Repeat Step 29 until the entire sample has passed through the RNeasy MinElute spin column. Discard the FT each time in a guanidinium waste container. (32) Add 500 μL RPE wash buffer to the RNeasy MinElute spin column. Centrifuge for 15 seconds at ≧8,000×g. Discard the FT. (33) Add 500 μL of 80% ethanol to the RNeasy MinElute spin column. Centrifuge for 15 seconds at ≧8,000×g. Discard the FT and the collection tube. (34) Place the RNeasy MinElute spin column in a new 2 ml collection tube. Centrifuge for 1 minute at ≧8,000×g. (35) Place the RNeasy MinElute spin column in a 1.5 mL collection tube. Add 14 μL of RNase-free water directly to the spin column membrane. Centrifuge for 1 minute at ≧8,000×g to elute the small RNA. Repeat with another 14 μL of RNase-free water. (36) Add 1 μL of RNase Block (Stratagene) and DTT to a final concentration of 10 mM. Store at −80° C. (37) Remove the columns containing the large RNAs from the temporary 4° C. storage. Add 700 μL RW1 buffer to the RNeasy column. (38) Centrifuge at ≧8,000×g. Discard FT and collection tube. (39) Transfer the RNeasy column to a new collection tube. (40) Pipette 500 μL of RPE buffer onto the RNeasy column.

(41) Centrifuge at ≧8,000×g, for 15 seconds. Discard FT. (42) Add another 500 μL of RPE buffer onto the RNeasy column. (43) Centrifuge for 2 minutes at ≧8,000×g to dry the RNeasy silica-gel membrane. (44) Transfer the RNeasy column to another tube and spin again as above for 1 minute to remove any traces of ethanol. (45) Transfer the RNeasy column to the final 1.5 mL collection tube. (46) Elute the large RNA fraction with 50 μL of RNase-free water. (47) Centrifuge at 8,000×g for 1 minute and then repeat elution with an additional 50 μL of RNase-free water. (48) Add 1 μL of RNase Block (Stratagene) and add DTT (final concentration of 10 mM) to the 100 μL final RNA sample. (49) Store at −80° C.

### Example 2: Preparation of Total Sperm mRNA Library (SpeRNA Library) and SpeRNA High-Throughput Sequencing

Total RNA (25 ng) prepared as described in the example above is used to prepare an mRNA library using the Illumina® TruSeq Stranded mRNA Library Prep Kit® for NeoPrep™. The SpeRNA library is then sequenced using one of the following systems: HiSeq 2500, HiSeq 3000, or HiSeq 4000 (Illumina®). Library preparation and sequencing are also offered as a service by commercial vendors, including Beckman Coulter. Accordingly, the methods may include issuing instructions to the vendor(s) to perform one or both of such tasks.

### Example 3: Synthesis of SpeRNA-Coded Peptides

Results from the previous step are run through the Basic Local Alignment Search Tool (BLAST). Specifically, the search is run against the Homo sapiens ESTs database using the Megablast algorithm for highly similar sequences, to identify sperm-expressed coding sequences.

For each identified gene, the corresponding protein sequence is retrieved, and then a library of overlapping peptides is created, using the following algorithm (amino acid positions are numbered from the N-terminus to the C-terminus of the protein): (A) Peptide #1 in the library corresponds to the first 15 amino acids; (B) peptide #2 starts at amino acid number 11 (which is included) and extends to amino acid number 25 (which is also included); and therefore peptides #1 and #2 overlap in 5 amino acids (C). In general, each peptide #n starts at the start position of peptide #(n−1)+10 and ends at the end position of peptide #(n−1)+10.

A 15 amino acid length distribution was applied due to the fact that the typical length distribution of antibody epitopes ranges from 5 to 15 amino acids (see, Singh, Ansari, et al. (2013)). Such a procedure will generate n=protein length/10 peptides for each identified proteins. Because a human protein is, on average, 480-amino acid in length, each peptide library consists of approximately 50 peptides. The human sperm proteome is estimated to include about 5,000 proteins (see, Wang, Guo, el al. (2013)). Therefore, a total of 250,000 peptides is expected. Customizable, high-density peptide chips that are commercially-available can contain up to 100,000 individual peptides per chip (see, Buus, Rockberg, et al. (2012)); therefore with only three of such high-density chips, the entire library can be screened.

In various embodiments of the present invention, each array is generated within a small (ca. 1×2 cm) rectangular area on a functionalized microscope slide. The individual peptide fields are square-shaped with a freely definable side length—varying from 10×10 μm and up to 1000×1000 μm. In various aspects, larger fields are used (e.g., 20×20 μm) fields to facilitate recording of the arrays after binding of ligands.

### Example 5: Analysis of Data for the Identification of Positive Peptides and Genes of Origin

Hybridization with sera and/or identification of immune-reactive peptides is performed. The entire process of hybridization and detection can be outsourced (e.g., Schafer-N ApS). As such, in some aspects, the methods include issuing instructions to perform such hybridization and/or identification.

Sera are analyzed from subjects newly diagnosed with solid or hematological malignancies. Each cancer diagnosis is analyzed separately. Normal control groups are different for each tumor group. Sera from cases (tumor diagnosed subjects) and controls (individuals with no known malignant condition) are collected by consecutive sampling. At least 100 cases and 100 controls are analyzed for each cancer type.

Intensities of signals for each peptide given by each serum are recorded. Values from the groups of both cases and controls are plotted separately and statistically significant differences are evaluated by the Mann-Whitney test (α=0.05). Before the analysis, outliers may be identified by the extreme studentized deviate (ESD) method or the Dixon's ratio test, and removed.

Peptides with calculated and those with a p<0.05 value are included in the new CTA panel and will undergo further testing by using: (i) Tetramer staining to identify naturally occurring, circulating, and/or tumor-infiltrating peptide-specific T-cells; (ii) cytotoxicity of in vitro generated CTL using autologous dendritic cell-PBL co-cultures; and/or (iii) in vivo murine models of the tumors of interest using murine homologues of the peptides sequence.

In T-cell based assays, each peptide can be used “as is” or corresponding T-cell specific, immune-dominant peptides can be derived from the protein containing the peptide originally identified by use of the present platform methodology disclosed herein.

Furthermore, the functional validation of identified antigen may be performed using the whole antigen, instead of the identified peptide(s). If more than one peptide is identified for each protein, the functional analysis will prioritize peptides with highest affinity score for the binding to class I MHC molecules, using the NetMHC 4.0 Server, for the prediction of peptide-MHC class I binding using artificial neural networks.

### 3. METHODOLOGY DEVELOPMENT AND RESULTS THEREFROM

By utilization of various methodologies which are disclosed and claimed in the present patent application, the Applicant, Kiromic BioPharma, Inc., has developed its' novel Biomarker Lead Antigen Discovery Engine (BLADE™) technology, which combines bio-informatics, high-throughput gene expression analysis, biochemistry, and immunology to identify, elucidate, and directly validate novel immune-target candidates in patients suffering from cancer.

Quantitative results from the utilization of these aforementioned methodologies are set forth in the Figures and will be discussed below.

### Selection of Cancer/Testis Antigens (CTAs)

Cancer/Testis Antigens (CTAs) are proteins that are expressed by tumors and a limited number of healthy and adult cell types. A defined number of CTAs is not expressed by healthy adult cell types (e.g., such as MAGE-A1, MAGE-C2, NY-ESO1). Thus, these CTAs are the ideal target of TCR therapies, because: (i) they are not expressed by healthy tissues, except testes and placenta, which do not express Major Histocompatibility (MHC) molecules and are thus undetected by T-cells (see, Hofmann O, Caballero O L, Stevenson B J, Chen Y T, Cohen T, Chua R, et al., “Genome-wide analysis of cancer/testis gene expression. Proc Natl Acad Sci USA (2008) 105(51):20422-7; (ii) CTAs are expressed by tumors of various histological origins (see, Id); and (iii) expression of CTAs has been associated with advanced stage disease and an unfavorable prognosis (see, Schuler-Thurne, B, Schultz E S, Berger T G, Weinlich G, Ebner S, Woer lP, et al., Rapid induction of tumor-specific type 1 T-helper cells in metastatic melanoma patients by vaccination with mature, cryopreserved, peptide-loaded monocyte-derived dendritic cells. J Exp Med (2002) 195(10):1279-88).

Therefore, the Applicant's T-cell therapy program aims to discover new T-cell targetable CTAs and their T-cell dominant epitope, to isolate and characterize powerful tumor-specific TCRs.

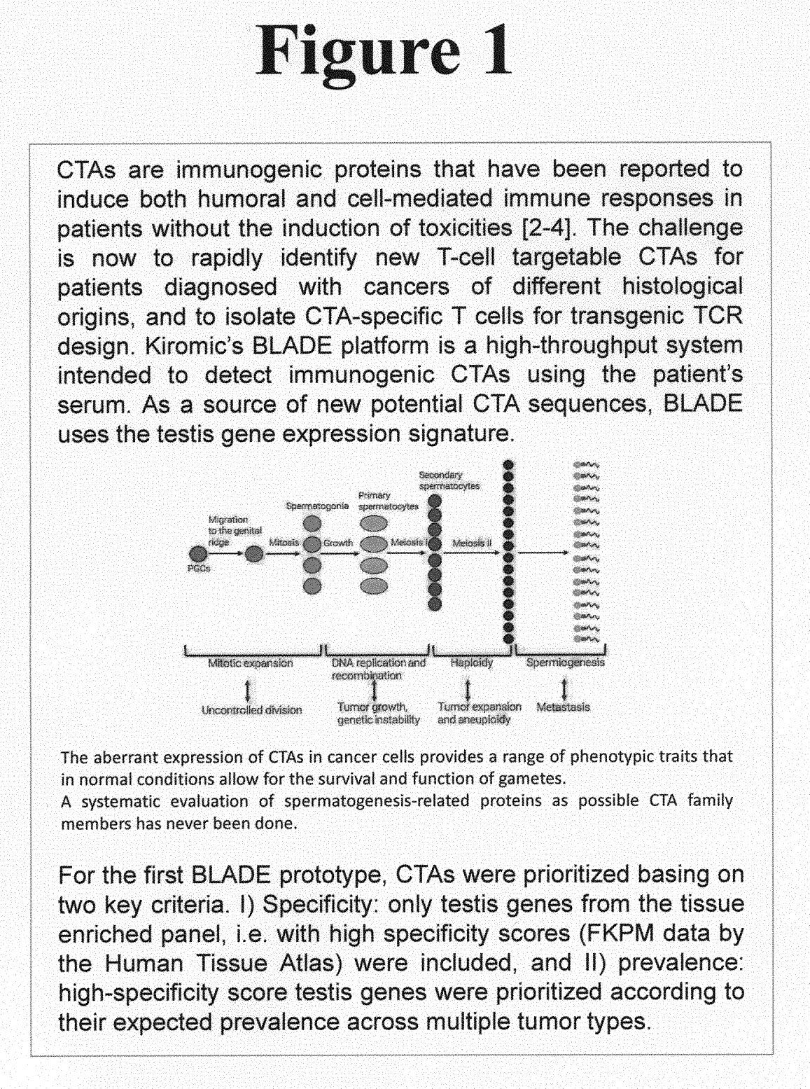

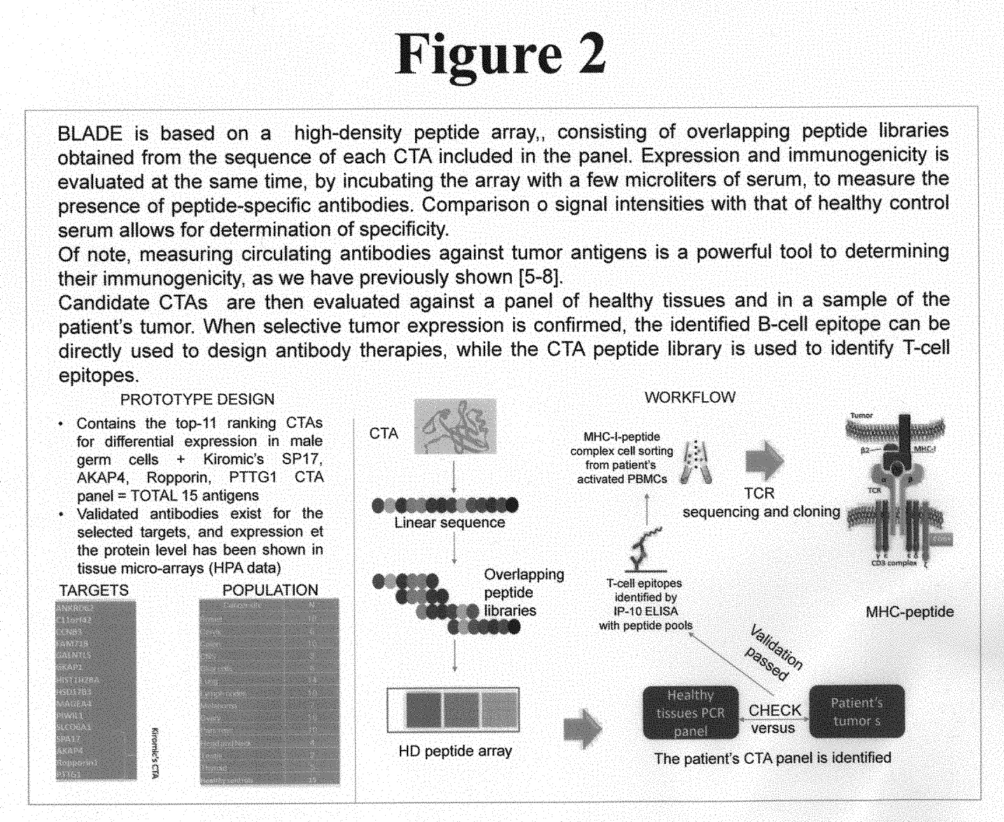

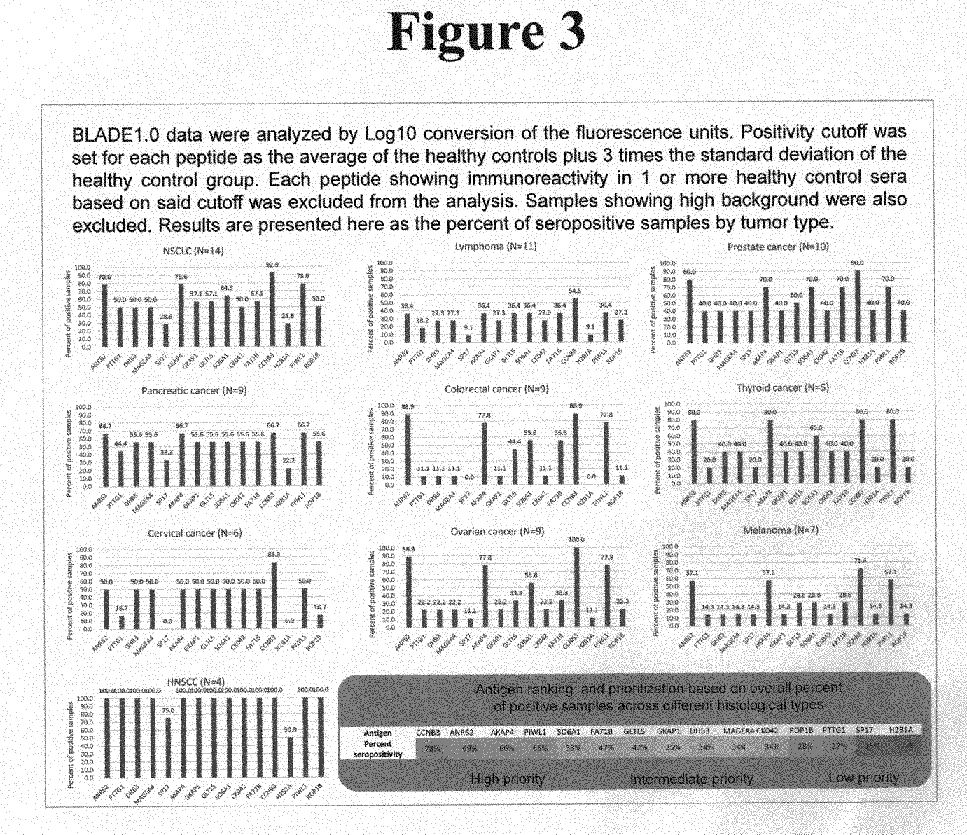

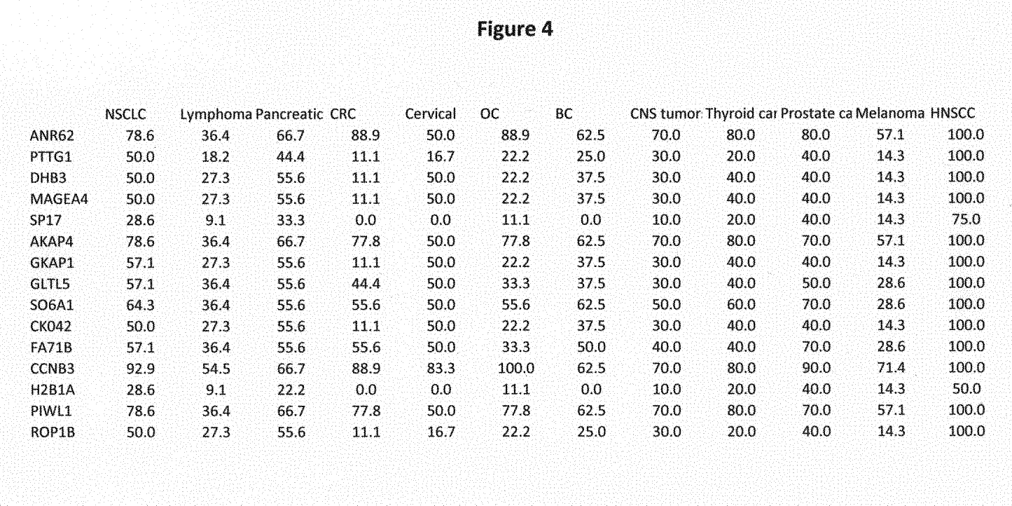

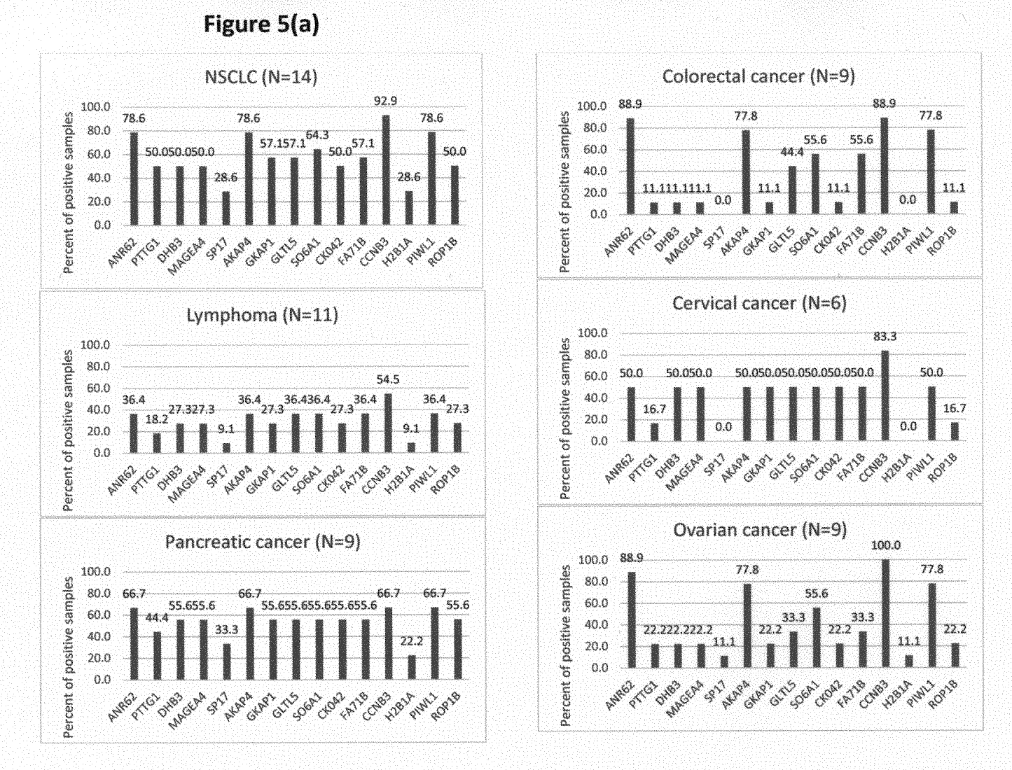

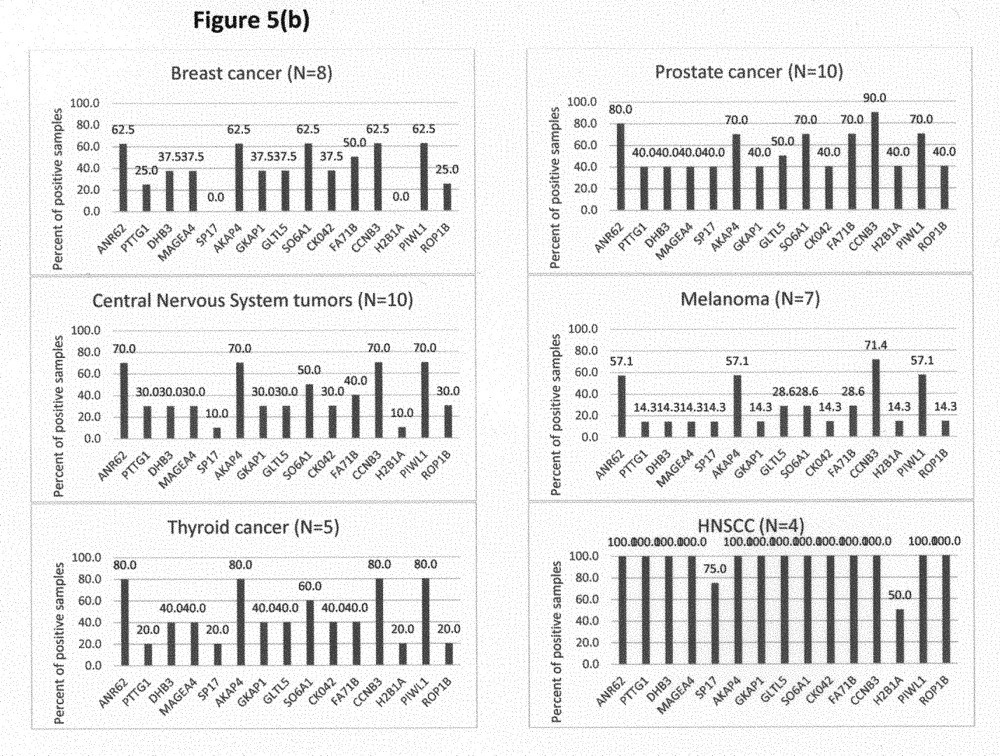

As set forth in FIG. 1, CTAs are immunogenic proteins that have been reported to induce both humoral and cell-mediated immune responses in patients without the induction of toxicities (see, Caballero O L, Chen Y T. Cancer/testis (CT) antigens: potential targets for immunotherapy. Cancer Sci (2009) 100(11):2014-21.doi:10.1111/j.1349-7006.2009.01303; Schuler-Thurner B, Schultz E S, Berger T G, Weinlich G, Ebner S, Woer lP, et al., Rapid induction of tumor-specific type 1 T-helper cells in metastatic melanoma patients by vaccination with mature, cryopreserved, peptide-loaded monocyte-derived dendritic cells. J Exp Med (2002) 195(10):1279-88; Lurquin C, Lethé B, De Plaen E, Corbière V, Théate I, van Baren N, et al., “Contrasting frequencies of antitumor and anti-vaccine T cells in metastases of a melanoma patient vaccinated with a MAGE tumor antigen.” J Exp Med (2005) 201(2):249-57). The challenge is now to rapidly identify new T-cell targetable CTAs for patients diagnosed with cancers of different histological origins, and to isolate CTA-specific T-cells for transgenic TCR design. The Applicant's have developed a novel selection platform (i.e., BLADE™) utilizing a high-throughput system intended to detect immunogenic CTAs using the patient's serum. As a source of new potential CTA sequences, BLADE™ uses the testis gene expression signature.

The aberrant expression of CTAs in cancer cells provides a range of phenotypic traits that in normal conditions allow for the survival and function of gametes. A systematic evaluation of spermatogenesis-related proteins as possible CTA family members has, heretofore, never been done.

For the first selection methodology (BLADE™ prototype), CTAs were prioritized basing upon two key criteria: (i) Specificity—only testis genes from the tissue enriched panel, i.e., with high specificity scores (FKPM data by the Human Tissue Atlas) were included; and (ii) Prevalence—high-specificity score testis genes were prioritized according to their expected prevalence across multiple tumor types.

The Applicant's approach to oncology is based upon the streamlined combination of its' highly novel and efficacious Biomarker Lead Antigen Discovery Engine (BLADE™) platform and its' ground-breaking Systemic Multi-Antigen Reprogrammed T-cell (SMART™) activation technology which, in general, is utilized in conjunction with the BLADE™ platform. This dual synergistic core technology allows Kiromic BioPharma, Inc. to uniquely integrate and optimize the two main steps in the design of cancer immunotherapies: (1) the discovery and validation of immune-targets (BLADE™), and (2) the safe and effective activation of the immune system by specifically reprogramming T-cells against cancer (SMART™).

The BLADE™ database consists of an optimized library of protein combinations derived from specific expression signatures. Through a proprietary protein array, BLADE™ is capable of directly detecting new, highly immunogenic novel immune-targets and concomitantly selecting the immune-dominant epitopes, all in a single step process. BLADE™ works with just a few drops of blood, and does not require a tumor sample; thus utilizing minimally-invasive procedures and producing very rapid results. Through its BLADE™ technology, Kiromic BioPharma, Inc. is able to identify novel cancer immunogenic peptides with a high probability of being translated into effective targets for cancer immunotherapy. Moreover, BLADE™ can also be utilized to detect and compare immunological signatures of cancer patients, so as to support the diagnosis, prognosis, and monitoring of immuno-targeted therapy responses. BLADE™ works with any cancer type and is, to date, the only known high-throughput platform for directly identifying new immune-targets based upon immunogenicity in a straightforward design for the development of novel cancer-targeted therapies.

In brief, Systemic Multi-Antigen Reprogrammed T-cell (SMART™) activation technology is a novel T-cell activation technology that combines the immune-targets elucidated by BLADE™ technology, and through a recombinant process of multifactorial peptides, results in a safe and effective activation of the immune system by specifically reprogramming T-cells against cancer.

### BLADE™ Platform Development

FIG. 2 sets forth the development of the Applicant's proprietary Biomarker Lead Antigen Discovery Engine (BLADE™) platform is based upon a high-density peptide array, consisting of overlapping peptide libraries obtained from the sequence of each CTA included in the panel. Expression and immunogenicity is evaluated at the same time, by incubating the array with a few microliters of serum, to measure the presence of peptide-specific antibodies. Comparison of signal intensities with that of healthy control serum allows for determination of specificity.

Of important note, the measurement of circulating (i.e., humoral) antibodies against tumor antigens is a powerful tool in determining their immunogenicity, as has previously been shown by the Inventors.

Candidate CTAs are then evaluated against a panel of healthy tissues and in a sample of the patient's tumor. When selective tumor expression is confirmed, the identified B-cell epitope can be directly used to design antibody therapies, while the CTA peptide library is used to identify T-cell epitopes.

### Exemplar Results

FIG. 3 graphically-illustrates: (i) the percent seropositivity of various samples by the type of tumor; and (ii) antigen-ranking and prioritization based upon overall percent of positive samples across different histological types.

BLADE™ data were analyzed by Log₁₀ conversion of the resulting measured fluorescence units. Positivity cutoff was set for each peptide, as the average of the healthy controls plus three-times the standard deviation of the healthy control group. Each peptide showing immunoreactivity in one or more healthy control sera, based on said cutoff, was excluded from the analysis. Samples showing high background were also excluded from the analysis.

Types of cancer tissues analyzed in these series of experiments include: (i) non-small cell lung carcinoma (NSCLC); (ii) lymphoma (iii) prostate; (iv) pancreatic; (v) colorectal; (vi) thyroid; (vii) cervical; (viii) ovarian; (ix) melanoma; and (x) head and neck squamous cell carcinoma (HNSCC).

FIG. 4 graphically-represents the percent of sero-positive cancer tissue samples. In the Table, columns represent the cancer types; whereas the rows identify the specific antigen type utilized.

FIGS. 5(a) and 5(b) graphically-illustrates the percent seropositivity of various samples by the type of tumor as screened by different antigens. BLADE™ data were analyzed by Log₁₀ conversion of the resulting measured fluorescence units. Positivity cutoff was set for each peptide, as the average of the healthy controls plus three-times the standard deviation of the healthy control group. Each peptide showing immunoreactivity in one or more healthy control sera, based on said cutoff, was excluded from the analysis. Samples showing high background were also excluded from the analysis.

Types of cancer tissues analyzed in these series of experiments include: (i) non-small cell lung carcinoma (NSCLC); (ii) lymphoma (iii) prostate; (iv) pancreatic; (v) colorectal; (vi) thyroid; (vii) cervical; (viii) ovarian; (ix) melanoma; (x) head and neck squamous cell carcinoma (HNSCC); and (xi) central nervous system.

### 4. UTILITY

Sperm-expressed proteins are a class of CTAs useful for developing cancer vaccines (Chiriva-Internati, Cobos, et al. (2008)). The disclosed subject matter provides high throughput techniques, which may be faster, cheaper, and/or more effective than existing methods for the identification of CTAs that may be clinically relevant as cancer biomarkers and immunotherapy targets. Also, the disclosed subject matter increases the number of available target antigens in cancer vaccines while also at adding knowledge on the relationship between human germ cell development and carcinogenesis.

### 5. EQUIVALENTS AND INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the disclosure.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. It is also to be understood that the terminology used herein is for the purpose of describing particular embodiments only, and is not intended to be limiting, since the scope of the present invention will be limited only by the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

### REFERENCES

Buus, S., et al. (2012). “High-resolution Mapping of Linear Antibody Epitopes Using Ultrahigh-density Peptide Microarrays.” Molecular & Cellular Proteomics: MCP 11(12): 1790-1800.
Caballero, O. L. and Y. T. Chen (2009). “Cancer/testis (CT) antigens: potential targets for immunotherapy.” Cancer Sci 100(11): 2014-2021.
Chiriva-Internati, M., et al. (2008). “Sperm fibrous sheath proteins: a potential new class of target antigens for use in human therapeutic cancer vaccines.” Cancer Immun 8: 8.
Gjerstorff, M. F., et al. (2015). “Oncogenic cancer/testis antigens: prime candidates for immunotherapy.” Oncotarget 6(18): 15772-15787.
Mellman, I., et al. (2011). “Cancer immunotherapy comes of age.” Nature 480(7378): 480-489.
Mirandola, L., et al. (2011). “Cancer testis antigens: novel biomarkers and targetable proteins for ovarian cancer.” Int Rev Immunol 30(2-3): 127-137.
Sammut, S. J., et al. (2014). “A novel cohort of cancer-testis biomarker genes revealed through meta-analysis of clinical data sets.” Oncoscience 1(5): 349-359.
Simpson, A. J., et al. (2005). “Cancer/testis antigens, gametogenesis and cancer.” Nat Rev Cancer 5(8): 615-625.
Singh, H., et al. (2013). “Improved method for linear B-cell epitope prediction using antigen's primary sequence.” PLoS One 8(5): e62216.
Wang, G., et al. (2013). “In-depth proteomic analysis of the human sperm reveals complex protein compositions.” J Proteomics 79: 114-122.

What is claimed is:

1. A method of identifying cancer/testes antigens (CTAs) for utilization as cancer treatment targets, said method comprising of identifying human sperm proteins to which patients, diagnosed with solid or hematological malignancies, have established a humoral immune response.

2. The method of claim 1, further comprising a preliminary step of isolating total sperm mRNA from a plurality of sperm cells.

3. The method of claim 2, further comprising sequencing the total sperm mRNA to generate total sperm mRNA sequencing data.

4. The method of claim 3, wherein the sequencing comprises performing whole exome sequencing (WES) on the plurality of sperm cells.

5. The method of claim 3, wherein the sequencing further comprises comparing the generated total sperm mRNA sequencing data to comprehensive human-expressed sequence data, which is stored in a database, so as to identify substantially-shared, sperm-expressed coding sequences as a target set of genes.

6. The method of claim 3, wherein when the total sperm mRNA sequencing data is not identical to the comprehensive sperm sequence data in an area of a substantially-shared, sperm-expressed coding sequence; and wherein the total sperm mRNA sequencing data is discarded and the comprehensive sperm sequence data for that area is retained as an identified target set of genes.

7. The method of claim 5, wherein the method further comprises immobilizing peptides corresponding with the target set of genes on a chip.

8. The method of claim 1, wherein the human sperm proteins are identified by the following steps:
(a) contacting an addressable array of peptide fragments representing all expressed human sperm protein sequences with serum from at least one cancer-surviving subject; and
(b) detecting specific binding of antibodies present within said at least one serum to one or more of the immobilized peptides.

9. The method of claim 8, further comprising the steps of:
(c) contacting the addressable array of peptide fragments with serum from at least one normal control subject, and
(d) detecting specific binding of antibodies present within said at least one serum to one or more of the immobilized peptides;
wherein the identified human sperm proteins are those for which specific binding of antibodies is greater in the cancer-surviving patient serum, than in the normal control subject serum.

10. The method of claim 9, wherein Step (c) is conducted concurrently with Step (a) and Step (c) is conducted concurrently with Step (b); wherein the antibodies of the serum isolated from cancer-surviving subject and antibodies of the serum isolated from the normal control subject are differentially labeled.

11. The method of claim 7, wherein the method further comprises screening the immobilized peptides with sera isolated from a cancer-surviving subject to produce a first signal and screening the immobilized peptides with sera isolated from a healthy subject to produce a second signal.

12. The method of claim 11, wherein comparing the first signal and second signal allows the ability to distinguish human sperm proteins to which patients diagnosed with solid or hematological malignancies have established a humoral immune response from human sperm proteins to which patients diagnosed with solid or hematological malignancies have not established a humoral immune response.

13. The method of claim 1, wherein the cancer/testes antigens (CTAs) are immune-reactive peptides.

14. The method of claim 1, wherein the cancer/testes antigens (CTAs) are sperm-expressed antigens.

15. A method of treating cancer in a subject, the method comprising:
(a) identifying cancer/testes antigens (CTAs) comprising human sperm proteins to which patients diagnosed with solid or hematological malignancies have established a humoral immune response;
(b) generating anti-cancer/testes antigens (CTA) polyclonal antibodies against the identified CTAs; and
(c) administering a therapeutically-effective amount of said antibodies to the subject and thereby eliciting an autologous immune response in the subject in a manner sufficient to treat the cancer.

16. The method of claim 15, wherein the cancer is a hematological malignancy.

17. The method of claim 16, wherein the hematological malignancy is selected from the group consisting of: T-cell acute lymphocytic leukemia, T-cell acute lymphoblastic leukemia, T-cell chronic lymphocytic leukemia, non-Hodgkin lymphomas, Hodgkin lymphoma, multiple myeloma, plasma cell leukemia, B-cell acute lymphocytic leukemia, B-cell acute lymphoblastic leukemia, chronic myelogenous leukemia (CML), and acute myeloid leukemia (AML).

18. The method of claim 15, wherein the cancer is a solid malignancy.
